(19) United States
(12) Patent Application Publication
(10) Pub. No.: US 2014/0254759 A1
(43) Pub. Date: Sep. 11, 2014

(54) IMAGING CONTROL APPARATUS, RADIOGRAPHIC IMAGING APPARATUS, RADIOGRAPHIC IMAGE CAPTURING SYSTEM, CONTROL METHOD, AND NON-TRANSITORY RECORDING MEDIUM STORING PROGRAM CAUSING COMPUTER TO EXECUTE CONTROL METHOD

(71) Applicant: CANON KABUSHIKI KAISHA, Tokyo (JP)

(72) Inventor: Tomohiko Haraguchi, Kawasaki-shi (JP)

(73) Assignee: CANON KABUSHIKI KAISHA, Tokyo (JP)

(21) Appl. No.: 14/198,269

(22) Filed: Mar. 5, 2014

(30) Foreign Application Priority Data
Mar. 6, 2013 (JP) 2013-044676

(51) Int. Cl. G01N 23/04

(52) U.S. Cl. CPC G01N 23/04
USPC 378/62

(57) ABSTRACT

An imaging control apparatus includes a determination unit and a setting unit. The determination unit is configured to determine, based on a state of communication to a radiation generator, which of a synchronous imaging mode and an asynchronous imaging mode is to be used to obtain radiographic image data. The synchronous imaging mode is a mode in which a radiographic imaging apparatus obtains radiographic image data by operating in synchronization with the radiation generator. The asynchronous imaging mode is a mode in which the radiographic imaging apparatus obtains radiographic image data without operating in synchronization with the radiation generator. The setting unit is configured to output an instruction for setting the imaging mode determined by the determination unit in the radiographic imaging apparatus such that the radiographic imaging apparatus obtains the radiographic image data in the imaging mode determined by the determination unit.

### BACKGROUND OF THE INVENTION

1. Field of the Invention

The present disclosure relates to a technique of setting an imaging mode of radiographic image data.

2. Description of the Related Art

Hitherto, X-ray images have been widely used in diagnosis of diseases and so on, and X-ray image capturing apparatuses that obtain digital X-ray image data by detecting X-rays radiated thereto have been developed. In particular, in recent years, portable X-ray image capturing apparatuses have become mainstream and applications of X-ray image capturing apparatuses based on wireless communication or the like is spreading.

In general, a portable X-ray image capturing apparatus includes a console used to control the entire system, an X-ray generator configured to radiate X-rays, and an X-ray interface configured to exchange signals or the like with the X-ray generator.

One of imaging modes employed by portable X-ray image capturing apparatuses to obtain X-ray image data is a synchronous imaging mode in which X-ray image data is obtained in a state where synchronization with the X-ray generator is achieved using the X-ray interface. The synchronous imaging mode is implemented through communication between the X-ray interface and the portable X-ray image capturing apparatuses. However, a difference between interfaces of the portable X-ray image capturing apparatuses and the X-ray generator sometimes makes it difficult to exchange signals and achieve synchronization. In such a case, an asynchronous imaging mode is available in which X-ray image data is obtained, for example, upon the portable X-ray image capturing apparatuses detecting X-rays, without achieving synchronization with the X-ray generator.

Hitherto, portable X-ray image capturing apparatuses supporting both the synchronous imaging mode and the asynchronous imaging mode have been developed. Such portable X-ray image capturing apparatuses use two consoles, namely, one for the synchronous imaging mode and one for the asynchronous imaging mode. However, recently, systems capable of handling both the synchronous imaging mode and the asynchronous imaging mode with one console have been developed.

In the case where an imaging process supporting both the synchronous imaging mode and the asynchronous imaging mode is performed using one console, the imaging mode is switched in accordance with a user operation. For the asynchronous imaging mode, there are some methods for detecting the start and end of imaging. However, depending on the method, there is a possibility that X-ray image data will not be successfully obtained. For this reason, in an environment where an imaging process in the synchronous imaging mode is permitted, the imaging process is desirably performed in the synchronous imaging mode whenever possible.

### SUMMARY OF THE INVENTION

According to some embodiments of the present invention, an imaging control apparatus includes a determination unit configured to determine, based on a state of communication to a radiation generator, which of a synchronous imaging mode and an asynchronous imaging mode is to be used to obtain radiographic image data, the synchronous imaging mode being a mode in which a radiographic imaging apparatus obtains radiographic image data by operating in synchronization with the radiation generator, the asynchronous imaging mode being a mode in which the radiographic imaging apparatus obtains radiographic image data without operating in synchronization with the radiation generator; and a setting unit configured to output an instruction for setting the imaging mode determined by the determination unit in the radiographic imaging apparatus such that the radiographic imaging apparatus obtains the radiographic image data in the imaging mode determined by the determination unit.

Further features of the present disclosure will become apparent from the following description of exemplary embodiments with reference to the attached drawings.

### BRIEF DESCRIPTION OF THE DRAWINGS

FIG. 1 is a diagram illustrating the configuration of an X-ray image capturing system according to first and second embodiments of the present invention.

FIG. 2 is a diagram illustrating a specific example of the configuration of the X-ray image capturing system according to the first embodiment of the present invention.

FIG. 3 is a sequence chart illustrating a process performed by the X-ray image capturing system according to the first embodiment of the present invention.

FIG. 4 is a diagram illustrating a specific example of the configuration of the X-ray image capturing system according to the second embodiment of the present invention.

FIG. 5 is a sequence chart illustrating a process performed by the X-ray image capturing system according to the second embodiment of the present invention.

FIG. 6 is a diagram illustrating the configuration of an X-ray image capturing system according to third and fourth embodiments of the present invention.

FIG. 7 is a diagram illustrating a specific example of the configuration of the X-ray image capturing system according to the third embodiment of the present invention.

FIG. 8 is a sequence chart illustrating a process performed by the X-ray image capturing system according to the third embodiment of the present invention.

FIG. 9 is a diagram illustrating the configuration of the X-ray image capturing system according to the fourth embodiment of the present invention.

FIG. 10 is a sequence chart illustrating a process performed by the X-ray image capturing system according to the fourth embodiment of the present invention.

### DESCRIPTION OF THE EMBODIMENTS

Exemplary embodiments of the present invention will be described in detail below with reference to the accompanying drawings. Note that embodiments to be described below are merely application examples of the present invention and the present invention is not limited to the embodiments below.

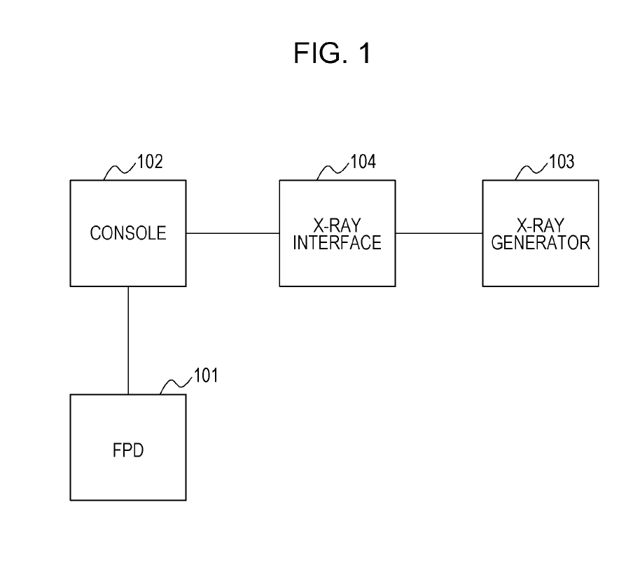

A first embodiment of the present invention will be described first. FIG. 1 is a diagram illustrating the configuration of an X-ray image capturing system according to the first embodiment. As illustrated in FIG. 1, an X-ray image capturing system according to the first embodiment includes a flat panel detector (FPD) 101 serving as a portable X-ray imaging apparatus, a console 102 used to control the X-ray image capturing system, an X-ray generator 103 configured to radiate X-rays, and an X-ray interface 104 configured control exchange of signals or the like between the console 102 and the X-ray generator 103. Note that the console 102 or the X-ray image capturing system is a component serving as an example of an imaging control apparatus. The X-ray generator 103 is a component serving as an example of a radiation generator. The first embodiment will be described regarding an example of obtaining X-ray image data. However, the present invention is also applicable to a case where radiographic image data is obtained using other kinds of radiation, such as alpha radiation and gamma radiation, as well as X-rays.

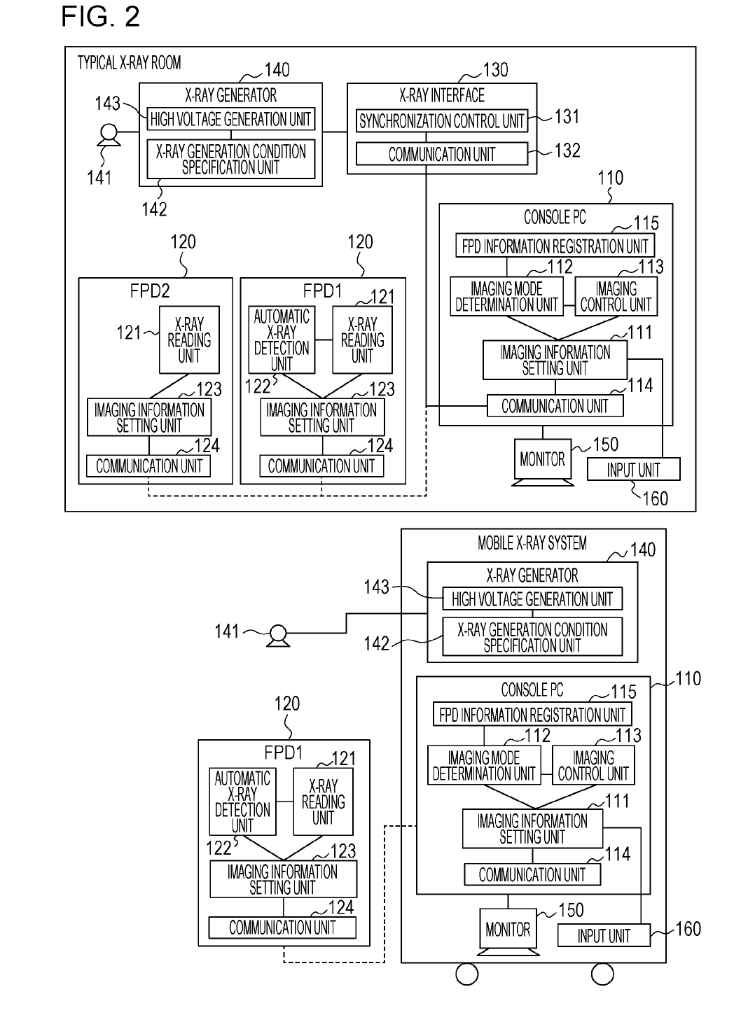

FIG. 2 is a diagram illustrating a specific example of the configuration of the X-ray image capturing system according to the first embodiment. The relationship between components illustrated in FIGS. 1 and 2 are as follows: the console 102 illustrated in FIG. 1 corresponds to a console personal computer (PC) 110 illustrated in FIG. 2; the X-ray interface 104 illustrated in FIG. 1 corresponds to an X-ray interface 130 illustrated in FIG. 2; the X-ray generator 103 illustrated in FIG. 1 corresponds to an X-ray generator 140 illustrated in FIG. 2; and the FPD 101 illustrated in FIG. 1 corresponds to each of FPDs 120 illustrated in FIG. 2.

First, the system configuration will be described using FIG. 2. The console PC 110 sets settings for communication with the FPD 120 and the X-ray generator 140, and controls operations of the FPD 120 and the X-ray generator 140. The console PC 110 includes an imaging information setting unit 111, an imaging mode determination unit 112, an imaging control unit 113, a communication unit 114, and an FPD information registration unit 115.

The imaging information setting unit 111 receives setting information that is input by the user via an input unit 160 and is to be used for communication with the FPD 120 and the X-ray interface 130, and stores the setting information in a memory. The imaging information setting unit 111 includes a flag which is switched depending on the imaging mode selected by the imaging mode determination unit 112 described below.

The imaging mode determination unit 112 is a program module that operates on a memory and performs the following procedure to determine whether or not imaging is to be performed in a state where synchronization with the X-ray generator 140 is achieved. The imaging mode determination unit 112 notifies the imaging information setting unit 111 of the selected imaging mode as its module output. In accordance with this output, the imaging information setting unit 111 switches the flag. An imaging mode selection method will be described in detail below.

The imaging control unit 113 transmits signals to and receives signals from the FPD 120 and the X-ray interface 130 so as to control the start and end of radiation of X-rays, the start and end of reading of an X-ray image, reception of the X-ray image, and so forth. In response to an operation performed on a program on a monitor 150 by a user, the imaging control unit 113 transmits an appropriate signal in a wired or wireless manner. In the case of synchronous imaging, the imaging control unit 113 transmits a signal triggering to start preparation of X-ray imaging to the FPD 120, receives a signal representing the completion of preparation of X-ray imaging from the FPD 120, and transmits a radiation permission signal to the X-ray interface 130. In the case of asynchronous imaging, communication between the console PC 110 and the X-ray interface 130 is not carried out. Thus, the X-ray generator 140 is permitted to radiate X-rays all the time. The FPD 120 starts capturing an image upon an automatic X-ray detection unit 122 detecting the radiated X-rays.

The communication unit 114 includes a communication circuit which includes an antenna unit and a communication control unit that actually perform communication and exchange signals with the FPD 120 and the X-ray interface 130 by using an Internet protocol (IP) address and a port number set by the imaging information setting unit 111. In the case of wired connection, transmission control protocol (TCP) communication is used to set imaging information or the like, and user datagram protocol (UDP) communication is used to transmit and receive signals and to transfer images or the like.

The FPD information registration unit 115 manages the registered FPDs 120 suitably used in the imaging mode selected by the imaging mode determination unit 112. FPD information is registered in the following manner. A wired or wireless connection is established to the FPD 120. The serial number, the model number, and so forth are received from the connected FPD 120. Then, an appropriate IP address is transmitted from the console PC 110 to the FPD 120. The registered FPD information is stored in a memory. Communication with the FPD 120 that has been registered before is restarted using the FPD information stored in the memory by re-establishing communication after disconnecting communication.

The FPD 120 is a radiographic imaging apparatus which is irradiated with radiation and obtains radiographic image data. The FPD 120 converts energy of X-rays radiated from the X-ray generator 140 into electric signals, constructs a digital X-ray image, and transfers the image to the console PC 110. The FPD 120 includes an X-ray reading unit 121, the automatic X-ray detection unit 122, an imaging information setting unit 123, and a communication unit 124.

The X-ray reading unit 121 includes a fluorescent material that coverts X-rays into visible light, and a sensor array that converts the visible light into electric signals. The X-ray reading unit 121 converts energy of received X-rays into electric charges, which are then accumulated in capacitors of pixels arranged in a matrix. The accumulated electric charges are subjected to analog-to-digital (A/D) conversion and are read as digital values via thin film transistor (TFT) switches and charge amplifiers. A TFT is a semiconductor element that enables a switching operation on a thin film transistor. By switching between ON and OFF of the TFT switches on a row-by-row basis, the X-ray reading unit 121 performs scanning to read pixels of the entire screen and obtain an X-ray image.

The automatic X-ray detection unit 122 includes a plurality of photo multipliers that are sensitive to X-rays, are arranged on the back side of the X-ray sensor array, and are connected to the X-ray reading unit 121. The automatic X-ray detection unit 122 detects the start or end of radiation based on signals supplied from the photo multipliers. In response to this detection, the X-ray reading unit 121 starts and ends X-ray reading. An FPD 1 includes the automatic X-ray detection unit 122, and thus is capable of performing asynchronous X-ray imaging. In contrast, an FPD 2 is only capable of performing synchronous X-ray imaging. Alternatively, the automatic X-ray detection unit 122 may detect the start of X-ray radiation by detecting a flow of current output from a power supply line of the above-described sensor array or scanning lines which transfer ON/OFF control signals from a driving circuit to the TFTs, for example.

The imaging information setting unit 123 writes and holds information used by the FPD 120 to communicate with the console PC 110 and the X-ray interface 130 in a field programmable gate array (FPGA). Also, the imaging information setting unit 123 receives the imaging mode selected by the imaging mode determination unit 112 from the console PC 110, and sets a setting as to whether or not to enable an asynchronous control flag of the FPGA in accordance with the imaging mode. If the asynchronous control flag is not enabled, the X-ray reading unit 121 operates in response to a radiation start signal received from the X-ray interface 130. On the other hand, if the asynchronous control flag is enabled, the X-ray reading unit 121 operates upon the automatic X-ray detection unit 122 detecting X rays.

The communication unit 124 is a communication circuit including an antenna and a communication control circuit, and actually exchanges information to be set by the imaging information setting unit 123 and so forth. The communication unit 124 actually performs communication and exchanges signals with the console PC 110 and the X-ray interface 130 by using an IP address and a port number set by the imaging information setting unit 123. In the case of wired connection, TCP communication is used to set imaging information and so forth, and UDP communication is used to transmit and receive signals, transfer images, and so forth.

The X-ray interface 130 is an apparatus independent of the console PC 110 and the FPD 120, is connected to a high voltage generation unit 143 of the X-ray generator 140, and controls X-ray radiation timings. The X-ray interface 130 includes a synchronization control unit 131 and a communication unit 132.

The synchronization control unit 131 controls the high voltage generation unit 143 connected thereto by a cable at a timing at which an X-ray radiation permission signal is received from the console PC 110 connected thereto by a cable. Upon receiving a radiation stop signal from the X-ray generator 140, the synchronization control unit 131 controls the high voltage generation unit 143 and transmits a radiation stop signal to the console PC 110.

The communication unit 132 is a communication circuit. The communication unit 132 stores, in a memory, IP addresses of the console PC 110 and the FPD 120 with which the communication unit 132 communicates, and transmits and receives signals using the information. With this configuration, an image can be captured in a state where synchronization is achieved between the FPD 120 and the X-ray generator 140.

Based on radiation conditions such as an X-ray tube voltage, an X-ray tube current, and an X-ray radiation duration set by an X-ray generation condition specification unit 142, the X-ray generator 140 supplies a high voltage generated by the high voltage generation unit 143 to an X-ray source 141 so as to radiate X-rays.

The console PC 110 is further connected to the monitor 150 and the input unit 160 including a keyboard, for example. The imaging information setting unit 111, the imaging mode determination unit 112, the imaging control unit 113, and the FPD information registration unit 115 described above are implemented by a central processing unit (CPU) of the console PC 110, programs executed by the CPU, a storage unit storing the programs, and a random access memory (RAM) to which instructions included in the programs are loaded. The instructions included in the programs are executed by the CPU, and the console PC 110 is controlled based on input/output information supplied from the communication unit 114, the input unit 160, and the monitor 150, whereby the configuration described above and processes described below are implemented. Similarly, the imaging information setting unit 123 of the FPD 120 is implemented by a CPU or the like of the FPD 120.

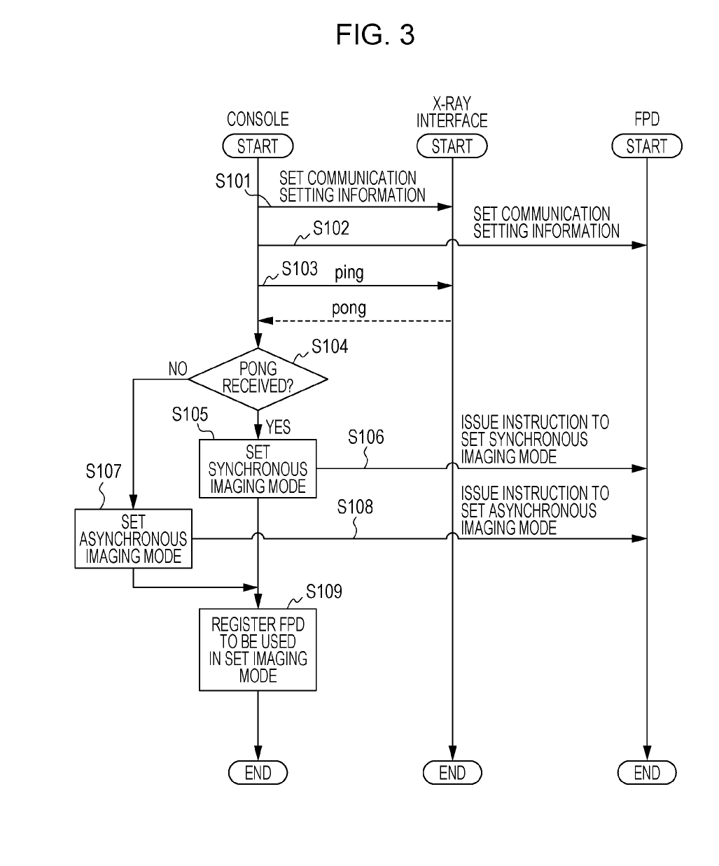

Referring next to FIG. 3, a process performed by the X-ray image capturing system according to the first embodiment will be described. Note that processing of steps S101 to S109 described below is processing implemented as a result of the CPU of the console 102 reading programs and data from a recording medium, such as a read only memory (ROM), and executing the programs.

In step S101, the console 102 sets setting information used for communication (hereinafter, referred to as communication setting information) in the X-ray interface 104. The communication setting information includes an IP address, a subnet mask, a default gateway, a port number, and so forth. When setting the communication setting information, the console 102 receives the communication setting information in response to a user operation, and determines whether or not the communication setting information is appropriate. If the communication setting information is appropriate, the console 102 transmits the communication setting information to the X-ray interface 104. Upon receiving the communication setting information from the console 102, the X-ray interface 104 sets the communication setting information therein. A specific setting method is as follows. A user inputs various pieces of setting information to the console 102 using the input unit 160. The imaging information setting unit 111 determines whether or not the input information is appropriate. If the input information is appropriate, the imaging information setting unit 111 transmits the information to the X-ray interface 104 via the communication unit 114. At this time, fixed IP addresses or the like may be set in advance for the FPD 101 and the X-ray interface 104.

In step S102, the communication unit 114 of the console 102 sets communication setting information in the FPD 101 as in step S101. Note that the order in which the communication setting information is set in the X-ray interface 104 and the FPD 101 is not limited to the above-described one. The communication setting information may be set in the X-ray interface 104 after the communication setting information is set in the FPD 101, or the communication setting information may be simultaneously set in the X-ray interface 104 and the FPD 101. Note that IP addresses may be set in advance for the FPD 101 and the X-ray interface 104.

In step S103, the imaging mode determination unit 112 of the console 102 transmits a ping to the IP address of the X-ray interface 104. In step S104, the console 102 determines whether it has received a pong. If a pong has been received, the imaging mode determination unit 112 determines that the X-ray interface 104 is connected to the console 102 and an imaging process in the synchronous imaging mode is available. In this case, the process proceeds to step S105. On the other hand, if a pong has not been received within a preset certain timeout period, the imaging mode determination unit 112 determines that the X-ray interface 104 is not connected to the console 102 and the synchronous imaging mode is not available. In this case, the process proceeds to step S107.

In the first embodiment, a method for detecting connection of the X-ray interface 104 through transmission of a ping and reception of a pong has been described. However, the method for detecting connection of the X-ray interface 104 employable in the present invention is not limited to this one. For example, when the imaging information setting unit 111 of the console 102 determines that the IP address of the X-ray interface 104 is not set in advance, the communication unit 114 of the console 102 makes an inquiry as to whether there is an X-ray interface ready for communication through broadcast. If three is an X-ray interface ready for communication, the X-ray interface returns information indicating that it is ready for communication to the console 102 in response to the inquiry. In this way, the imaging mode determination unit 112 of the console 102 determines that an imaging process in the synchronous imaging mode is available.

In step S105, the imaging information setting unit 111 of the console 102 sets the synchronous imaging mode therein. As a result of this setting, the imaging control unit 113 issues an instruction to enter an X-ray radiation waiting state to the FPD 101 when the console 102 receives an X-ray radiation instruction from a user. After entering the X-ray radiation waiting state in response to the instruction from the console 102, the FPD 101 transmits a radiation permission signal to the X-ray generator 103 and obtains X-ray image data. Thereafter, when receiving an X-ray radiation end instruction from a user or when a radiation duration has reached the maximum radiation period, the FPD 101 causes the X-ray generator 103 to end X-ray radiation, and transfers the resulting X-ray image data to the console 102.

In step S106, the console 102 issues an instruction to set the synchronous imaging mode to the imaging information setting unit 123 of the FPD 101. In this way, the synchronous imaging mode is set in the FPD 101, and the FPD 101 becomes ready to obtain X-ray image data in response to an instruction to enter the X-ray radiation waiting state received from the console 102.

In step S107, the imaging information setting unit 111 of the console 102 sets the asynchronous imaging mode therein. In step S108, the console 102 instructs the FPD 101 to set the asynchronous imaging mode therein. In this way, the asynchronous imaging mode is set in the FPD 101. By performing the above-described procedure during the startup of the system using the console 102, the imaging mode flag of the imaging information setting unit 111 is set. Also, by regularly repeating the above-described procedure, the configuration of the system is monitored and the imaging mode flag is automatically changed when the configuration of the system is changed.

After the switching to the synchronous imaging mode or the asynchronous imaging mode is completed in the above-described manner, the console 102 registers the FPD 101 to be used in the set imaging mode in step S109 in response to a user operation. At this time, when the user attempts to register the FPD 101 not supporting the set imaging mode, the console 102 notifies the user that the FPD 101 to be registered does not support the set imaging mode and thus is unable to capture an image. Alternatively, the imaging mode switched by the console 102 may be changed in accordance with the user operation.

The FPD 101 supporting the synchronous imaging mode and the asynchronous imaging mode may be registered by the FPD information registration unit 115 of the console 102 before the console 102 transmits a ping. In this case, the console 102 registers the FPD 101 supporting the imaging modes in response to a user operation. Then, the console 102 switches the imaging mode in accordance with a result of ping transmission by performing processing similar to that of steps S103 to S108 of FIG. 3. At this time, if the registered FPD 101 does not support the set imaging mode, the console 102 notifies the user that the FPD 101 does not support the set imaging mode. Alternatively, in this case, the imaging mode switched by the console 102 may be changed in accordance with the user operation.

In the first embodiment, the console 102 determines whether or not the X-ray interface 104 is connected thereto and switches the imaging mode in accordance with the obtained result. In this way, the first embodiment makes it possible to automatically set an appropriate imaging mode to obtain X-ray image data, without requiring any user operation. Also, in the case where the FPD 101 does not support the set imaging mode, the user is notified of this state. This consequently prevents obtaining of X-ray image data using the FPD 101 not supporting the set imaging mode from occurring.

A second embodiment of the present invention will be described next. In the first embodiment, the console 102 determines whether or not the X-ray interface 104 is connected thereto and switches the imaging mode in accordance with the obtained result. In contrast, in the second embodiment, the FPD 101 determines whether or not the X-ray interface 104 is connected thereto and switches the imaging mode in accordance with the obtained result. An X-ray image capturing system according to the second embodiment has a configuration similar to that illustrated in FIG. 1, and thus the references used in FIG. 1 are also used in a description of the second embodiment.

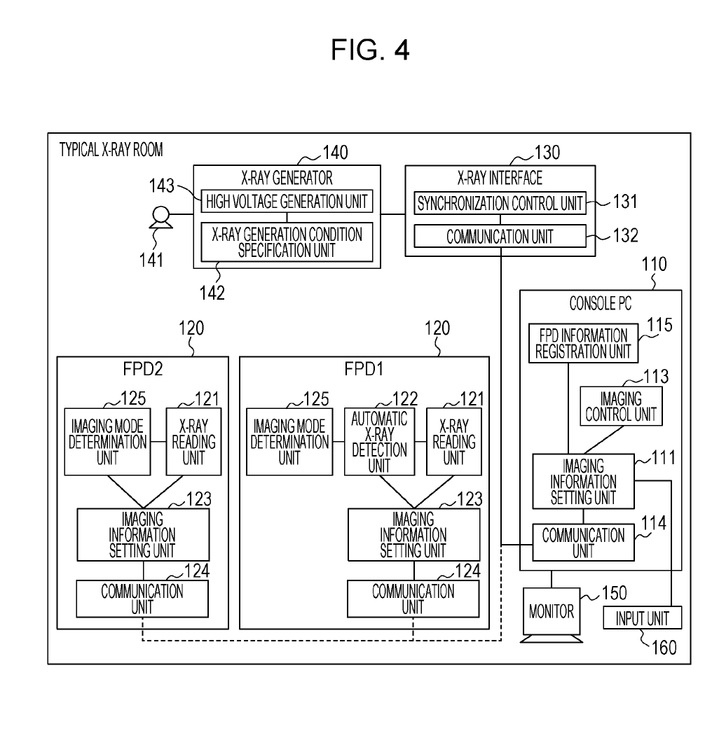

The configuration of the X-ray image capturing system according to the second embodiment will be described below with reference to FIG. 4. The system configuration is similar to that of the first embodiment described above. In the above-described first embodiment, the console PC 110 includes the imaging mode determination unit 112. In contrast, in the second embodiment, the FPD 120 includes an imaging mode determination unit 125 configured to detect connection of the X-ray interface 130 and select the imaging mode.

The imaging mode determination unit 125 is a program module that operates on the FPGA and performs the following procedure to determine whether or not imaging is to be performed in a state where synchronization with the X-ray generator 140 is achieved. The imaging mode determination unit 125 notifies the imaging information setting unit 123 of the selected imaging mode as its module output. In accordance with this output, the imaging information setting unit 123 switches the imaging mode flag. An imaging mode selection method will be described in detail below.

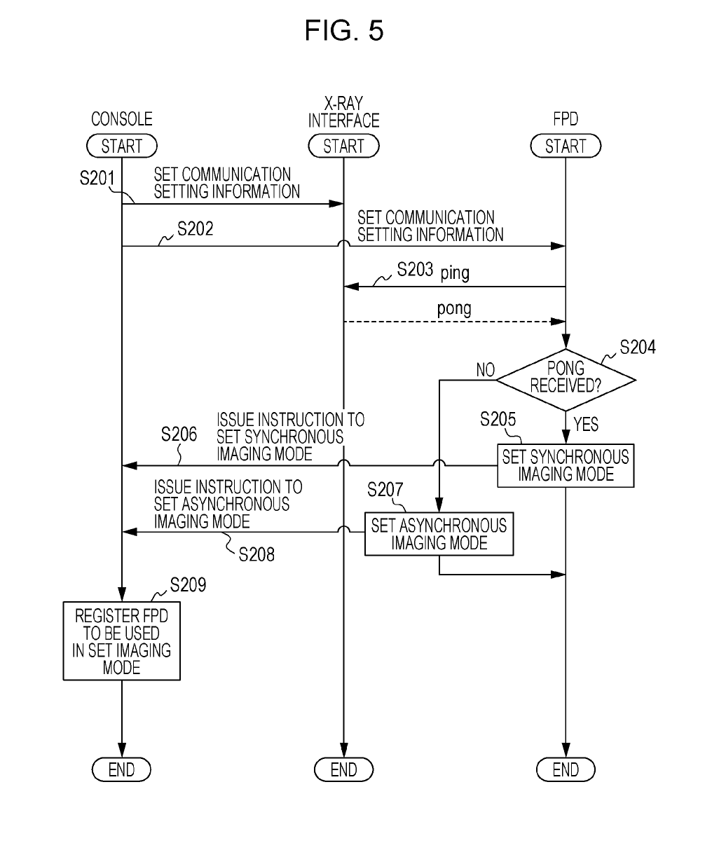

Referring to FIG. 5, a process performed by the X-ray image capturing system according to the second embodiment will be described. Note that processing of steps S201, S202, and S209 of FIG. 5 is processing implemented as a result of the CPU of the console 102 reading programs and data from a recording medium, such as a ROM, and executing the programs. Also, processing of steps S203 to S208 of FIG. 5 is processing implemented as a result of the CPU of the FPD 101 reading programs and data from a recording medium, such as a ROM, and executing the read programs.

In step S201, the console 102 sets communication setting information in the X-ray interface 104 as in step S101 of FIG. 3 of the first embodiment. In step S202, the console 102 sets communication setting information in the FPD 101 as in step S102 of FIG. 3 of the first embodiment. At this time, the console 102 sets the IP address of the X-ray interface 104 in the FPD 101. Note that the order in which the communication setting information is set in the X-ray interface 104 and the FPD 101 is not limited to the above-described one, as in the first embodiment.

In step S203, the FPD 101 transmits a ping to the X-ray interface 104. In step S204, the FPD 101 determines whether or not it has received a pong. If a pong has been received, the FPD 101 determines that the X-ray interface 104 is connected thereto and the synchronous imaging mode is available. In this case, the process proceeds to step S205. On the other hand, if a pong has not been received within a preset certain timeout period, the FPD 101 determines that the X-ray interface 104 is not connected thereto and the synchronous imaging mode is not available. In this case, the process proceeds to step S207.

As in the first embodiment, the method for detecting connection of the X-ray interface 104 through transmission of a ping and reception of a pong has been described also in the second embodiment. However, the method for detecting connection of the X-ray interface 104 employable in the present invention is not limited to this one. When the IP address of the X-ray interface 104 is not set in the FPD 101, the FPD 101 makes an inquiry as to whether there is an X-ray interface ready for communication through broadcast. If there is an X-ray interface ready for communication, the X-ray interface returns information indicating that it is ready for communication to the FPD 101 in response to the inquiry. In this way, the FPD 101 determines that an imaging process in the synchronous imaging mode is available.

In step S205, the FPD 101 sets the synchronous imaging mode therein. As a result of this setting, the FPD 101 becomes ready to obtain X-ray image data in response to receipt of an instruction to enter the X-ray radiation waiting state from the console 102.

In step S206, the FPD 101 instructs the console 102 to set the synchronous imaging mode therein. Consequently, the console 102 instructs, upon receiving an X-ray radiation instruction from a user, the FPD 101 to enter the X-ray radiation waiting state. After entering the X-ray radiation waiting state in response to the instruction received from the console 102, the FPD 101 transmits a radiation permission signal to the X-ray generator 103 and obtains X-ray image data. Thereafter, when receiving an X-ray radiation end instruction from a user or when a radiation duration has reached the maximum radiation period, the FPD 101 causes the X-ray generator 103 to end X-ray radiation, and transfers the resulting X-ray image data to the console 102.

In step S207, the FPD 101 sets the asynchronous imaging mode therein. In step S208, the FPD 101 instructs the console 102 to set the asynchronous imaging mode therein. In this way, the asynchronous imaging mode is set in the console 102.

After the switching to the synchronous imaging mode or the asynchronous imaging mode is completed in the above-described manner, the console 102 registers the FPD 101 to be used in the set imaging mode in step S209 in response to a user operation. At this time, when the user attempts to register the FPD 101 not supporting the set imaging mode, the console 102 may notify the user that the FPD 101 to be registered does not support the set imaging mode and thus is unable to capture an image, as in the first embodiment.

In the second embodiment, the FPD 101 detects whether or not the X-ray interface 104 is connected thereto and switches the imaging mode in accordance with the obtained result. In this way, the second embodiment makes it possible to obtain X-ray image data in an appropriate imaging mode, without requiring any user operation. Also, in the case where the FPD 101 does not support the set imaging mode, the user is notified of this state in step S209. This consequently prevents obtaining of X-ray image data using the FPD 101 not supporting the set imaging mode from occurring.

A third embodiment of the present invention will be described next. In the third embodiment, an example will be described in which a console and an FPD are capable of performing wireless communication with each other via an access point.

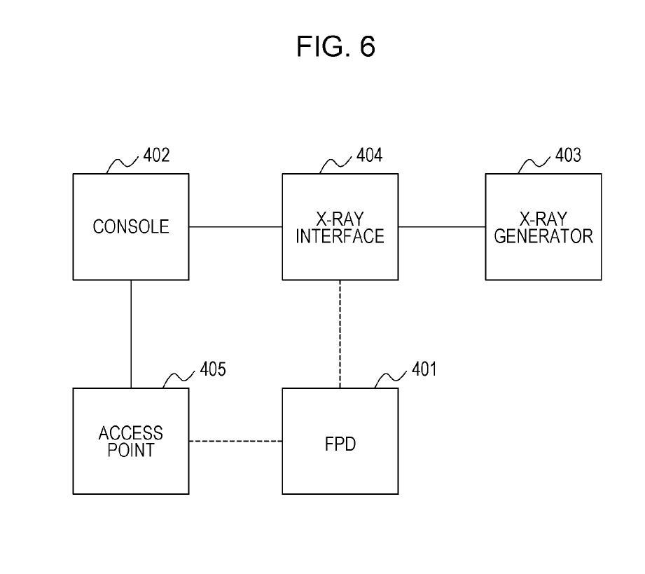

FIG. 6 is a diagram illustrating the configuration of an X-ray image capturing system according to the third embodiment. As illustrated in FIG. 6, the X-ray image capturing system according to the third embodiment includes an FPD 401 which is a portable X-ray imaging apparatus capable of performing wireless communication, a console 402 used to control the X-ray image capturing system, an X-ray generator 403 configured to radiate X-rays, an X-ray interface 404 configured to control exchange of signals or the like between the console 402 and the X-ray generator 403, and an access point 405 configured to relay wireless communication between the FPD 401 and the console 402.

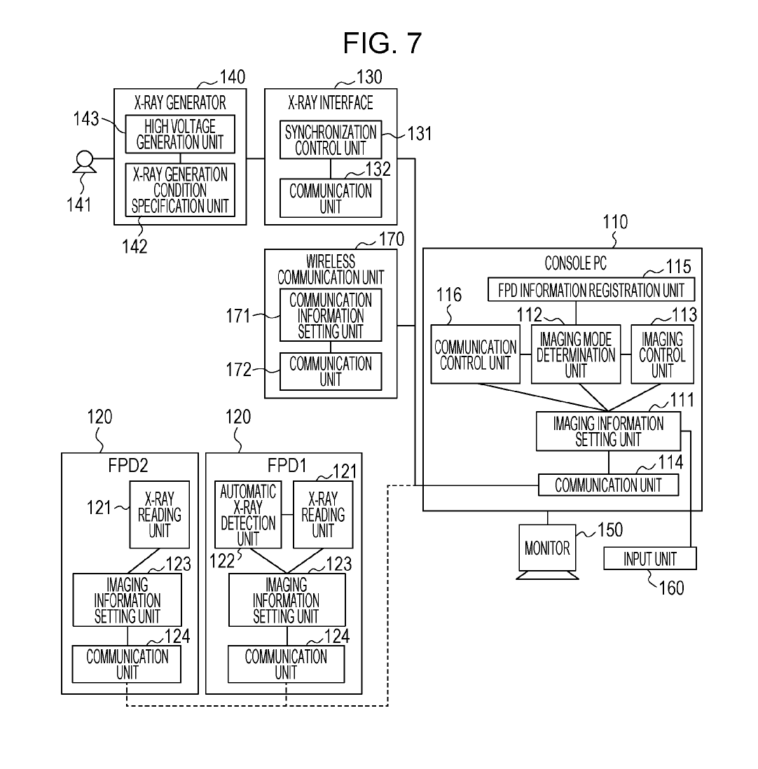

FIG. 7 is a diagram illustrating a specific example of the above-described configuration. The FPD 401 illustrated in FIG. 6 corresponds to each of the FPDs 120 illustrated in FIG. 7. The console 402 illustrated in FIG. 6 corresponds to the console PC 110 illustrated in FIG. 7. The X-ray generator 403 illustrated in FIG. 6 corresponds to the X-ray generator 140 illustrated in FIG. 7. The X-ray interface 404 illustrated in FIG. 6 corresponds to the X-ray interface 130 illustrated in FIG. 7. The access point 405 illustrated in FIG. 6 corresponds to a wireless communication unit 170 illustrated in FIG. 7. In the third embodiment, there is provided the wireless communication unit 170 serving as a relay to be used to perform communication between the console PC 110 and the FPD 120.

The wireless communication unit 170 includes a communication information setting unit 171 and a communication unit 172. The wireless communication unit 170 is a typical stationary wireless communication unit and refers to a radio wave relay configured to connect terminals to each other using a wireless local area network (LAN). The wireless communication unit 170 generally has a function for connection to a wired LAN, and is connected to the console PC 110, and is used as a relay in wireless communication to the FPD 120.

The communication information setting unit 171 stores information used to perform wireless communication with the FPD 120 in a memory included in the wireless communication unit 170. Typically, the communication information setting unit 171 is notified, via wired connection, of information set in the console PC 110 by a user using the input unit 160.

The communication unit 172 exchanges signals using the information stored by the communication information setting unit 171 via an antenna unit included therein and a cable connected to the console PC 110.

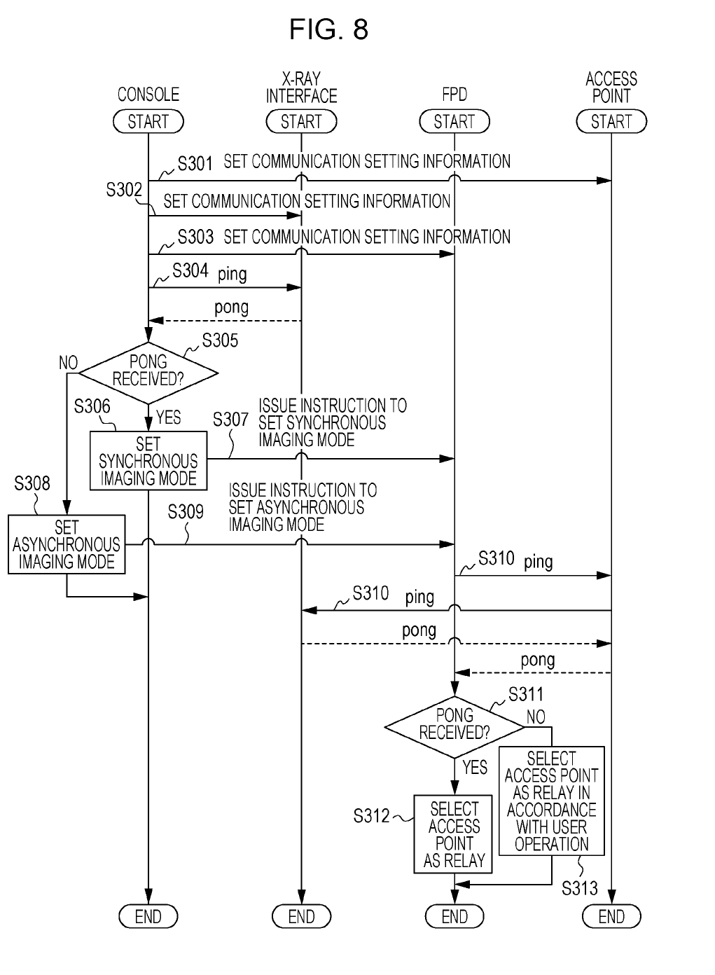

Referring to FIG. 8, a process performed by the X-ray image capturing system according to the third embodiment will be described below. Note that processing of steps S301 to S309 of FIG. 8 is processing implemented as a result of the CPU of the console 402 reading programs and data from a recording medium, such as a ROM, and executing the programs. Also, processing of steps S310 to S313 of FIG. 8 is processing implemented as a result of the CPU of the FPD 401 reading programs and data from a recording medium, such as a ROM, and executing the programs.

In step S301, the console 402 sets communication setting information in the access point 405. The communication setting information includes an IP address of the access point 405, ID information for identifying the access point 405, encryption information, a communication band to be used, and so forth. When setting the communication setting information in the access point 405, the console 402 receives the communication setting information in response to a user operation, and transmits the communication setting information to the access point 405 via TCP/IP communication or the like. In this way, the communication setting information is set in the access point 405. Note that in the case where a plurality of access points 405 are connected, the console 402 sets the communication setting information in each of the access points 405.

In step S302, the console 402 sets communication setting information in the X-ray interface 404 as in step S101 of FIG. 3 of the first embodiment. In step S303, the console 402 sets communication setting information in the FPD 401 as in step S102 of FIG. 3 of the first embodiment. At this time, the console 402 notifies the FPD 401 of information regarding the access point 405 to be used. In this way, the FPD 401 becomes able to perform wireless communication with the console 402 via the access point 405. Note that the order in which the communication setting information is set in the X-ray interface 404 and the FPD 401 is not limited to the above-described one, as in the first and second embodiments. The information regarding the access point 405 which the FPD 401 is notified of includes the ID of each access point 405, an IP address of the access point 405 corresponding to the ID, and so forth. In the case where a plurality of access points 405 are connected, the FPD 101 is notified of information regarding all the access points 405. Note that processing of steps S304 to S309 of FIG. 8 is similar to that of steps S103 to S108 of FIG. 3, and thus a description thereof is omitted.

In step S310, the FPD 401 transmits, via the access point 405, a ping to the IP address of the X-ray interface 404 which the FPD 401 is notified of by the console 402. In step S311, the FPD 401 determines whether or not it has received a pong within a timeout period. If the FPD 401 has received a pong from the X-ray interface 404 (if the synchronous imaging mode is set in steps S306 and S307), the process proceeds to step S312. On the other hand, if the FPD 401 has not received a pong from the X-ray interface 404 (if the asynchronous imaging mode is set in steps S308 and S309), the process proceeds to step S313. In step S312, the FPD 401 selects the access point 405 as a relay (relay apparatus) used to perform wireless communication with the X-ray generator 403 via the X-ray interface 404. In step S313, the FPD 401 selects the access point 405 selected in accordance with a user operation as a relay used to perform wireless communication with the X-ray generator 403 via the X-ray interface 404.

In this way, third embodiment makes it possible to appropriately select the access point 405 to be used to communicate with the X-ray interface 404, without requiring any user operation, and consequently improve the operation efficiency. Note that processing for registering the FPD 401 performed in step S109 of FIG. 3 may be added after the processing of steps S306 and S307 or steps S308 and S309. In the third embodiment, the console 402 determines whether communication to the X-ray interface 404 is connected and sets the imaging mode in accordance with the obtained result. However, this function may be executed by the FPD 401 as in the second embodiment.

A fourth embodiment of the present invention will be described next. In the first to third embodiments, whether or not the X-ray interface 404 is connected is detected, and the imaging mode is switched in accordance with the obtained result. In contrast, in the fourth embodiment, whether or not an access point serving as a relay in wireless communication is connected is detected, and the imaging mode is switched in accordance with the obtained result. The fourth embodiment will be described below using the case where there are a plurality of access points which include both a hardware access point and a software access point by way of example.

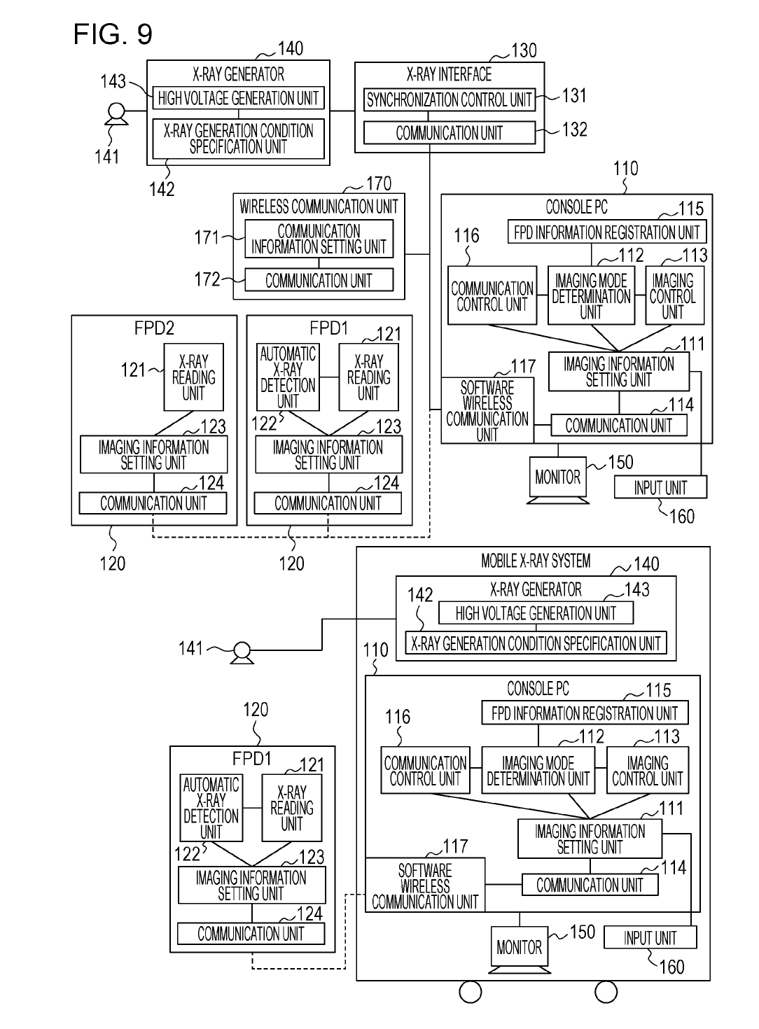

The configuration of an X-ray image capturing system according to the fourth embodiment will be described with reference to FIG. 9. Unlike the example illustrated in FIG. 7, an example illustrated in FIG. 9 includes the wireless communication unit 170 which is a hardware access point constituted by dedicated hardware, and a software wireless communication unit 117 which is a software access point constituted by software programs executed by the communication circuit and the CPU. Here, a hardware access point is a stationary access point and refers to a relay used to connect terminals to each other via a wireless LAN. The hardware access point generally has a function for connection to a wired LAN, is connected to the console 402, and is used as a relay in wireless communication to the FPD 401. On the other hand, a software access point is implementation of functions of the hardware access point by software and allows a computer or the like to function as the hardware access point. Technologies such as virtual Wi-Fi and tethering have been developed as representative software access points. These technologies can change the system configuration from the one requiring both the computer and the hardware access point to the one including only the computer, and consequently can improve the portability of the X-ray image capturing system.

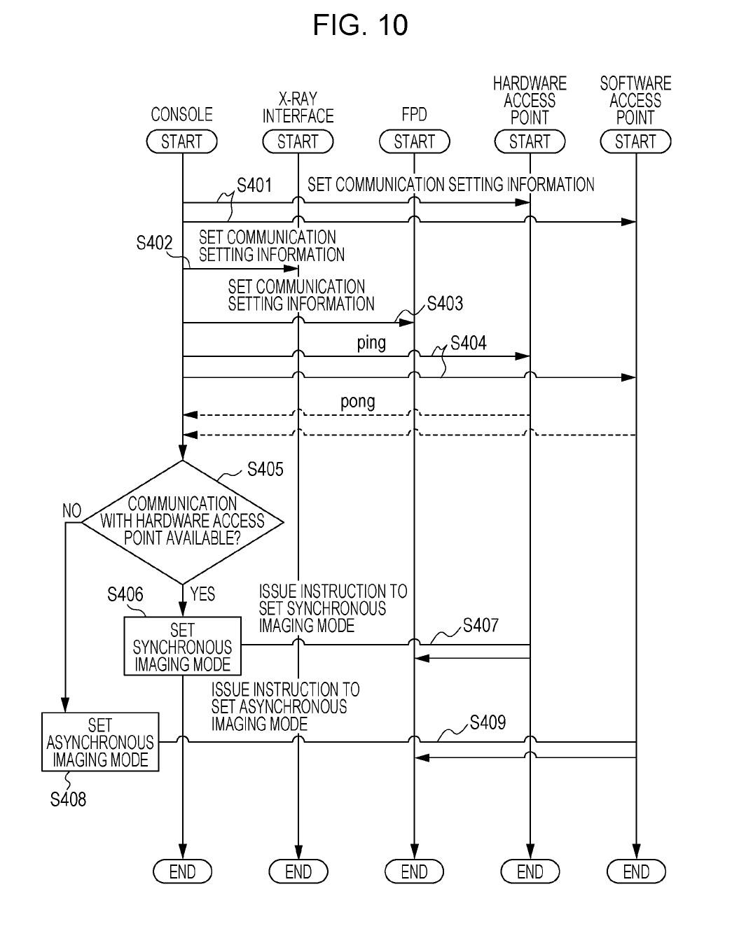

Referring to FIG. 10, a process performed by the X-ray image capturing system according to the fourth embodiment of the present invention will be described below. Note that processing of steps S401 to S409 of FIG. 10 is processing implemented as a result of the CPU of the console 402 reading programs and data from a recording medium, such as a ROM, and executing the programs.

In step S401, the console 402 sets communication setting information in the access points 405 as in step S301 of FIG. 8 of the third embodiment. Like the hardware access point, the software access point requires an IP address and information for identifying the access point. Accordingly, by setting information for enabling identification of the hardware access point and the software access point, such as the IP address and the ID, whether the access point 405 used by the console 402 and the FPD 401 is a hardware access point or a software access point can be identified. Examples of a method for identifying whether an access point of interest is a hardware access point or a software access point include adding an identifiable keyword to an EEID of the access point.

In step S402, the console 402 sets communication setting information in the X-ray interface 404 as in step S302 of FIG. 8 of the third embodiment. In step S403, the console 402 sets communication setting information in the FPD 401 as in step S303 of FIG. 8 of the third embodiment.

In step S404, the console 402 transmits a ping to the hardware access point and the software access point. In step S405, the console 402 determines whether it has received a pong from the hardware access point and the software access point within a timeout period. If the console 402 has received a pong from the hardware access point, the console 402 determines that communication to the hardware access point is available. In this case, the process proceeds to step S406. On the other hand, if the console 402 has not received a pong from the hardware access point but has received a pong from the software access point, the console 402 determines that only communication to the software access point is available. In this case, the process proceeds to step S408.

In step S406, the console 402 sets the synchronous imaging mode therein. In step S407, the console 402 instructs, via the hardware access point, the FPD 401 to set the synchronous imaging mode therein. Note that in the case where there are a plurality of hardware access points, an access point capable of communicating with the X-ray interface 404 is selected as in the third embodiment.

In step S408, the console 402 sets the asynchronous imaging mode therein. In step S409, the console 402 instructs, via the software access point, the FPD 401 to set the asynchronous imaging mode therein. Note that in the case where there are a plurality of software access points, a given software access point may be selected from among the plurality of software access points in accordance with a user operation. Note that processing for registering the FPD 401 performed in step S109 of FIG. 3 may be added after the processing of steps S406 and S407 or steps S408 and S409.

In the fourth embodiment, the method for switching the imaging mode to the synchronous imaging mode in response to detection of communication to the hardware access point has been described. However, in another embodiment, the imaging mode may be switched to the synchronous imaging mode in response to detection of communication to the software access point. Alternatively, the user may be allowed to select the hardware access point or the software access point so that imaging mode is to be switched to the synchronous imaging mode in response to detection of communication to the selected access point.

In the fourth embodiment, when communication to the hardware access point is available, the synchronous imaging mode is selected in order to implement stable imaging process and communication process, without considering the portability of the X-ray image capturing system. When only communication to the software access point is available, the asynchronous imaging mode is selected by placing importance on the portability of the X-ray image capturing system by minimizing the number of necessary apparatuses. This configuration enables construction of a system that gives high satisfaction to users.

The embodiments of the present invention can be implemented as a result of execution of the following process. Specifically, software (program) implementing the functions of the above-described embodiments is supplied to a system or apparatus via a network or various types of recording media. A computer (or a CPU, micro processing unit (MPC), or the like) of the system or apparatus reads and executes the program.

According to the above-described embodiments of the present invention, an appropriate imaging mode can be automatically set.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory apparatus, a memory card, and the like.

While the present disclosure has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-044676, filed Mar. 6, 2013, which is hereby incorporated by reference herein in its entirety.

What is claimed is:

1. An imaging control apparatus comprising:
a determination unit configured to determine, based on a state of communication to a radiation generator, which of a synchronous imaging mode and an asynchronous imaging mode is to be used to obtain radiographic image data, the synchronous imaging mode being a mode in which a radiographic imaging apparatus obtains radiographic image data by operating in synchronization with the radiation generator, the asynchronous imaging mode being a mode in which the radiographic imaging apparatus obtains radiographic image data without operating in synchronization with the radiation generator; and
a setting unit configured to output an instruction for setting the imaging mode determined by the determination unit in the radiographic imaging apparatus such that the radiographic imaging apparatus obtains the radiographic image data in the imaging mode determined by the determination unit.

2. The imaging control apparatus according to claim 1, wherein the determination unit is configured to determine, based on whether or not communication to the radiation generator is established, which of the synchronous imaging mode and the asynchronous imaging mode is to be used to obtain the radiographic image data.

3. The imaging control apparatus according to claim 1, further comprising
a registration unit configured to register a radiographic imaging apparatus that is to be used in the imaging mode set by the setting unit and is configured to obtain radiographic image data.

4. The imaging control apparatus according to claim 3, further comprising
a notification unit configured to notify, in a case where the radiographic imaging apparatus to be registered by the registration unit does not support the imaging mode set by the setting unit, a user that the radiographic imaging apparatus does not support the imaging mode.

5. The imaging control apparatus according to claim 1, wherein the imaging control apparatus is included in the radiographic imaging apparatus configured to obtain radiographic image data.

6. The imaging control apparatus according to claim 1, further comprising
a selection unit configured to select, based on whether or not wireless communication to the radiation generator is established via a relay apparatus, the relay apparatus as a relay apparatus to be used to perform wireless communication between the radiographic imaging apparatus and the radiation generator.

7. The imaging control apparatus according to claim 1, wherein the determination unit is configured to determine, based on a type of a relay apparatus to be used to perform wireless communication between the radiographic imaging apparatus and the radiation generator, which of the synchronous imaging mode and the asynchronous imaging mode is to be used to obtain the radiographic image data.

8. The imaging control apparatus according to claim 7, wherein the determination unit is configured to determine, if the relay apparatus is a hardware access point, the synchronous imaging mode is to be used to obtain the radiographic image data, and determine, if the relay apparatus is a software access point, the asynchronous imaging mode is to be used to obtain the radiographic image data.

9. A radiographic imaging apparatus comprising:
a determination unit configured to determine, based on a state of communication to a radiation generator, which of a first imaging mode and a second imaging mode is to be used to obtain radiographic image data, the first imaging mode being a mode in which the radiographic imaging apparatus obtains radiographic image data by performing synchronous communication with the radiation generator, the second imaging mode being a mode in which the radiographic imaging apparatus obtains radiographic image data without performing synchronous communication with the radiation generator; and
a control unit configured to perform control so as to obtain the radiographic image data in the imaging mode determined by the determination unit.

10. A radiographic image capturing system comprising:
a radiographic imaging apparatus;
a radiation generator;
a determination unit configured to determine, based on a state of communication to the radiation generator, which of a synchronous imaging mode and an asynchronous imaging mode is to be used to obtain radiographic image data, the synchronous imaging mode being a mode in which the radiographic imaging apparatus obtains radiographic image data by operating in synchronization with the radiation generator, the asynchronous imaging mode being a mode in which the radiographic imaging apparatus obtains radiographic image data without operating in synchronization with the radiation generator; and
a setting unit configured to set a setting in the radiographic imaging apparatus such that the radiographic imaging apparatus obtains the radiographic image data in the imaging mode determined by the determination unit.

11. A control method comprising:
determining, based on a state of communication to a radiation generator, which of a synchronous imaging mode and an asynchronous imaging mode is to be used to obtain radiographic image data, the synchronous imaging mode being a mode in which a radiographic imaging apparatus obtains radiographic image data by operating in synchronization with the radiation generator, the asynchronous imaging mode being a mode in which the radiographic imaging apparatus obtains radiographic image data without operating in synchronization with the radiation generator; and
setting a setting in the radiographic imaging apparatus such that the radiographic imaging apparatus obtains the radiographic image data in the imaging mode determined by the determination unit.

12. A non-transitory recording medium storing a program causing a computer to execute the control method according to claim 11.
